(19) United States
(12) Patent Application Publication
(10) Pub. No.: US 2024/0328935 A1
(43) Pub. Date: Oct. 3, 2024

(54) Method and system for monitoring the quality of a fermentation process for beer

(71) Applicant: Manfred Dausch, Göcklingen (DE)

(72) Inventor: Manfred Dausch, Göcklingen (DE)

(21) Appl. No.: 18/579,418

(22) PCT Filed: Jul. 15, 2022
(86) PCT No.: PCT/EP2022/069918

§ 371 Date: Jan. 15, 2024

(30) Foreign Application Priority Data
Jul. 16, 2021 (DE) 102021118440.1

(51) Int. Cl. G01N 21/33; C12C 11/00; G01N 1/40; G01N 33/14

(52) U.S. Cl. CPC G01N 21/33; C12C 11/003; G01N 1/4077; G01N 33/146; G01N 2001/4083

(57) ABSTRACT

Methods and systems for automated quality monitoring of the fermentation process and/or for determining the end point of fermentation during the beer brewing process in the production of beer by determining one or more aroma components or other ingredients of the beer.

### TECHNICAL FIELD

The present invention relates to a method and a plant for automated quality monitoring of the fermentation process and/or for determining the end point of fermentation during the beer brewing process in the production of beer by determining one or more aroma components or other ingredients of the beer.

### STATE OF THE ART

The brewing process for the production of beer is based on a fermentation, during which several metabolic processes take place that lead to the formation of various chemical compounds and by-products. During alcoholic fermentation, the metabolism of the beer yeast used for fermentation produces several by-products that substantially influence the aroma, flavor and therefore also the quality of the beer. These by-products include among others vicinal diketones (VDK), higher alcohols, esters, organic acids, sulfur compounds, phenols, carbonyls, and hops oil. Therefore, a beer is a highly complex multi-component mixture with a large number of aroma compounds, the presence and concentration of which can eventually be attributed to the complex yeast metabolism. In a broad sense, there are the bouquet substances, which include higher alcohols, esters, and organic acids. They basically determine the aroma of the beer. In addition, there are also so-called young beer bouquet substances, which include aldehydes, vicinal diketones and sulfur compounds. If present, they provide the beer with an unripe, disharmonious flavor or smell and, in higher concentrations, they have a negative effect on the beer quality. The aim of fermentation and the beer maturation process is to reduce the concentration of young beer bouquet substances in the beer as far as possible and to accumulate the bouquet substances in the desired concentration ranges.

Until now, it has been impossible to specifically control all the substances responsible for the beer aroma in the fermentation and maturation process.

Thus, lead substances that correlate with the sensory quality of the beer are used to determine the success of fermentation and the degree of maturation of beer. These lead substances include the vicinal diketones, which give the beer a foul, sweetish and, at higher concentrations, even disgusting flavor when a certain threshold is exceeded. The most important aroma components of vicinal diketones include diacetyl (butane-2,3-dione) and pentane-2,3-dione. In the beer brewing process, beer yeasts produce diacetyl from the metabolic intermediate acetolactate, which they further reduce to 2,3-butanediol. During fermentation, the concentrations of free diacetyl are usually low and α-acetolactate constitutes the majority of the “total diacetyl” present. Therefore, in the analysis, diacetyl concentrations are often expressed as “total diacetyl” concentrations, i.e. as the total amount of free diacetyl and α-acetolactate (“potential diacetyl” or “diacetyl precursor”), to emphasize potential diacetyl concentrations.

The aroma of diacetyl is often described as buttery, sweet, and occasionally as caramelly. The concentration of vicinal diketones in finished beer (i.e., in filled bottles or kegs) is therefore a key criterion for the degree of maturation and thus for the quality of the beer. Diacetyl has a flavor threshold of approximately 0.1 to 0.15 milligrams per liter, 2,3-pentandione has a threshold approximately 10 times higher.

The monitoring of vicinal diketones as lead substances and other aroma components is therefore an important feature during the fermentation process. Preferably, the threshold is between 3 and 100 ppb (parts per billion), wherein also intermediate ranges and intermediate values are covered by the invention. For light lager beers, the threshold of diacetyl is usually between 5 and 20 ppb. For dark beers, a higher threshold between 40 and 100 ppb, preferably between 60 and 80 ppb, is often sufficient due to the human sensory threshold.

People's perception of diacetyl in beer is quite different and sensory differences are often genetically determined. However, a large number of people are sensitive to flavor and notice changes in flavor due to higher concentrations of vicinal diacetones in finished beer.

The vicinal diketones are not produced by the yeast itself in its metabolism, but only their precursors. The precursors are acetohydroxy acids that are released into the beer during the fermentation process. The precursors of vicinal diketones do not have any flavor or smell and can therefore only be determined using sensitive gas chromatography methods. The precursor of diacetyl (butane-2,3-dione, 2,3-butanedione) is 2-acetolactate, the precursor of pentane-2,3-dione (2,3-pentanedione) is 2-acetohydroxybutyrate. During the fermentation process, the concentrations of the precursors of the vicinal diketones are increasing, with their peak usually being reached between 20 and 50% of the fermentation time, depending on the type of beer. The precursors are then converted into vicinal diketones, the concentration of which is highest at the end of fermentation. During the subsequent maturation process of the beer during tank storage, the VDK levels fall again as the yeast uses the VDK as an energy source. If the yeast is removed too early during the maturation process or the process is stopped, VDK can later occur in the finished beer and cause the undesirable loss of flavor. For that reason, brewers monitor the VDK levels during the beer maturation process. For traditional lager beer, the lowest possible VDK value is preferred.

Therefore, the beer is stored for several weeks after fermentation so that the yeast has sufficient time to metabolize the remaining VDK.

Until now, attempts have been made to control the VDK levels by specifically selecting the fermentation parameters and thus the quantity of acetohydroxy acids formed during fermentation. These parameters include the selection of the yeast strain or yeast mutants, the concentration of yeast during fermentation, the input of oxygen, the wort composition, and finally also the fermentation temperature and pressure.

Besides that, there are also methods in which the temperature is measured during the fermentation process in order to estimate when the precursors of VDK are converted to VDK based on the temperature changes. However, this method is very inaccurate, as it is not possible to determine when the yeast has metabolized the remaining VDK.

By using chemical-physical methods, it is possible to detect aroma components and other ingredients via their signatures. Methods such as ion mobility spectrometry (IMS) with an upstream gas chromatography (GC) column (GC-IMS) or gas chromatography with electron capture detector (GC-ECD) are used to determine the ingredients. However, these are laboratory methods that are very time-consuming and costly. This is because only random samples are taken, which have to be sent to a laboratory in order to be analyzed. Not every brewery has its own laboratory. However, one laboratory is often responsible for several breweries. Sometimes, it can take several days before the results arrive. During this time, the fermentation vessels have to be further cooled, which is associated with high costs and capacity problems. In addition, the time for maturing the beer has often already been exceeded and the quality of the beer is no longer optimal. Such a time-delayed measurement for determining VDK in the laboratory is therefore very problematic. as, for example, there is no opportunity to intervene in the event of faulty batches. Furthermore, errors can occur during sampling (e.g., due to oxidation), during sample transportation (e.g., oxidation, duration, temperature) or during sample preparation (degassing and filtration). Finally, the entire fermentation process, which can last more than 10 days, is usually monitored with only one or two measurements, which leads to very long downtimes for the beers in the individual tanks. This in turn results in enormous capacity problems for the breweries.

WO 2017/218039 A1 describes an in-line method for detecting chemical compounds in beer. In this method, the chemical compounds are measured by means of an infrared (IR) cell using IR spectroscopy. The chemical compound can be a vicinal diketone. The IR cell can be an IR-ATR cell, in which, for example, RAMAN-spectroscopy, gas electrode analysis, headspace analysis by gas chromatography or electrochemical detection can be used. The beer sample is pumped into a heater in order to obtain IR-ATR data. Based on the calculated diacetyl compound concentrations. the fermentation parameters can be adapted. However, in this method, an IR spectroscopy has to be conducted, which is correspondingly time-consuming and prone to failure. Also, the fermentation success is ultimately not reliable and therefore the degree of maturation of beer during the brewing process.

In conclusion, there is currently no reliable method to fully monitor the fermentation success and thus the degree of maturation of beer during the beer brewing process in an online process.

In order to no longer operate the maturation process “blindly”, it is therefore desirable for the breweries to use a method or system that enables a fully automated quality control for the beer and thus the degree of maturation of the beer.

### DESCRIPTION OF THE INVENTION

It is thus the object of the present invention to provide a method and a system for the automated quality monitoring of the fermentation success and the degree of maturation of beer during the beer brewing process, with which it is possible to determine components of the beer, in particular aroma components of the beer, during the process (online process) without having to perform a manual sample taking.

This object is solved by a method with the features of claim 1 and a plant for performing such a method. Preferred embodiments can be found in the subclaims.

An important feature of the method or plant according to the invention can be seen in the sample preparation, which has a significant impact on the measurement result and thus on the evaluation. According to the invention, the sample preparation consists of degassing an extracted sample to remove non-liquid suspended matter. For this purpose, a degassing module with a subsequent filtration device for removing suspended matter (e.g., yeast, cell residues) from the beer sample can be provided. Alternatively, degassing takes place during centrifugation. The filtrate or centrifugate is then fed into a sample conversion module and the samples are heated until the precursors of the aroma components have been completely converted into the actual aroma components.

The method according to the invention is designed for automated quality monitoring of the fermentation process and/or for determining the end point of fermentation during the beer brewing process in the production of beer and is based on an automated determination of components of the beer, preferably aroma components of the beer, in particular vicinal diketones (VDK), but also other ingredients such as aldehydes, sulfur compounds or organic compounds. The object is to obtain an in-line control of the beer quality in the process in order to optimize the fermentation and maturation process in the production of beer. The required storage times can thus be precisely determined for each type of beer. It is also possible to immediately detect a faulty batch. By automating quality monitoring, process steps can be immediately implemented as the end point of fermentation can be precisely determined. Up to now, it was necessary to await the laboratory results until the concentrations of the flavoring agents were available. This often takes up to 24 hours, as many large breweries measure the diacetyl and 2,3-pentanedione values of the flavoring agents several times a day to determine the perfect value for the end of the fermentation in the so-called “cold block”. So far, the values have been analyzed in the laboratory using a gas chromatograph (GC) and an ECD detector. As measurements are usually only taken once a day, it is very difficult to say exactly when which tank is “ripe” for bottling. This process is also very time-consuming and labor intensive.

The method according to the invention, however, can automatically measure at certain times, for example every 20 minutes, and determines the desired values at a low detection limit of diacetyl and 2,3-pentanedione of 1 ppb (parts per billion). If the VDK concentration is too high and the desired beer quality has not been achieved yet, the maturing process can be extended. Vice versa, the maturing process can be stopped when the desired beer quality has been achieved. Thus, the end point of the fermentation or maturation process is determined within a very short time using the in-line determination method according to the invention. The method thus enables automated quality monitoring of the fermentation process and/or determination of the end point of fermentation during the beer brewing process during production. Tracking the fermentation process and the maturation process to determine the degree of maturation of the beer is easily possible. The method according to the invention is based, among other things, on the determination of one or more aroma components of the beer, in particular vicinal diketones, such as diacetyl (butane-2,3-dione) or pentane-2,3-dione, or other ingredients that are characteristic of the respective beer. According to the invention, sample preparation is necessary before the measurement in order to be able to obtain reliable measurement results during the fermentation process. Sample preparation includes, among other things, degassing the beer sample extracted from the fermentation vessel, removing suspended matter if necessary, converting ingredients in the sample containers and extracting sample gas from the sample containers in order to then feed it into a measuring device.

Determining the concentration of VDK is particularly necessary in order to determine the end point of fermentation. The further or other components or ingredients of the beer can be analyzed with the measuring device in order to obtain an impression of the fermentation process and/or the quality of the beer. Preferably, this is done by spectroscopic analysis of one or more ingredients of the beer and a subsequent principal component analysis.

The method according to the invention for automated quality monitoring of the fermentation process and/or for determining the end point of fermentation during the beer brewing process in the production of beer by determining one or more aroma components or other ingredients of a beer, comprises in a preferred embodiment the following steps:
a) automated extraction of a beer sample from one or more fermentation vessels,
b) if required, degassing and filtering or centrifuging of the beer sample to remove non liquid suspended matter,
c) feeding the filtrate or the centrifugate of the beer sample into a sample conversion module consisting of one or more sample containers, into each of which a defined sample volume of the filtered or centrifuged beer sample is fed as required, which has a sufficient headspace volume,
d) heating the samples in the sample container or containers to convert precursors of the aroma components into the aroma components or to convert ingredients, wherein the heating is performed until the precursors of the aroma components have been almost completely converted into the aroma components,
e) extracting sample gas from the headspace of the sample container or from one of the sample containers and transferring the sample gas to a measuring device for the qualitative and quantitative determination of one or more components of the beer.

Filtration or centrifugation is preferably performed in a separation device. If the extracted beer sample is filtered, degassing of the sample is required for an optimal result. Therefore, after the automated extraction of the beer sample from the one or more fermentation containers, the method according to the invention comprises transferring the sample extracted from the fermentation container(s) in a degassing module and degassing it. Not until then is the beer sample filtered to remove non-liquid suspended matter, if required.

When using a centrifuge, for example a vertical decanter centrifuge, the degassing module can also be dispensed. In this case, the beer sample is directly led into the centrifuge from the fermentation container(s). After centrifugation, the centrifugate of the beer sample is fed into the sample conversion module.

“Periodic extraction” refers to the extraction of a beer sample from a tank farm at fixed times or intervals. The extraction is automated by using and arranging process valves, which enable specific sample volumes to be taken from the fermentation vessels.

Suspended matters (also known as turbidity) are known in the beer brewing process. These include, for example, chemical (e.g. proteins) and biological (e.g. yeasts) suspended matters of other suspended matters.

In large breweries, fermentation usually takes place in tank farms or fermenters, whereby alcoholic fermentation needs several days. The amount of diacetyl in the fermentation vessel is influenced by the fermentation temperature, the fermentation time and the pH value, among other factors. As the VDK are converted Into less flavor-intensive molecules towards the end of the fermentation process, the beer should only remain in the vessels for as long as necessary to achieve a higher efficiency of beer production and to ensure the quality of the beer product. By extracting a sample from the tank in a first process step, a beer sample is automatically extracted from the fermentation vessel or fermentation tank in a defined cycle and the VDK concentration is determined. For this purpose, several valves are switched for sampling, eliminating the need for manual sampling.

In an optional process step, the sample extracted from the tanks is transferred to a degassing module and then to an optional separation device, e.g. a filter, a filter device or a centrifuge. Sampling valves on the tank and sample lines are provided for sampling. The separation device is used to remove suspended matter that could impair the analytical evaluation when determining the VDK. Particles can interfere with the measurement by scattering or absorbing light. However, a separation device may not be necessary for certain types of beer.

The separation device or filtration module preferably comprises a filter unit, a filtrate container, an optional cooling system as well as pumps for fluid delivery. In another preferred embodiment, it is provided that the filter can be backflushed after each measurement. Therefore, a backflushable filter is preferably used.

The term “filtration” should initially be interpreted in a broad sense, as it includes the use of a separation device, preferably a filter device. Alternatively, solid particles and suspended matter are removed by centrifugation, preferably a decanter centrifuge. This treatment produces either a clear filtrate or centrifugate. This sample preparation is necessary as gas bubbles or particles would interfere with subsequent analysis, for example in GC-IMS or UV/VIS spectroscopy, Also, gas bubbles should not form in the event of a pressure drop. A head of foam, in tum, could interfere with the phase transition during gas phase extraction. Preferably, filters with a pore size of 1-3 μm are used for filtration. Preferably, the filter has a structure that allows particles with a diameter of >2 μm to be filtered out of the beer sample.

For degassing, the beer sample is first transported by the pre-pressure to the degassing module with minimal foam formation and the vessel is then depressurized. The beer sample is preferably degassed by constant stirring so that it can leave the degassing module free of gas and foam. The degassing module preferably comprises a degassing vessel and preferably an agitator arranged therein for performing the degassing. Preferably, degassing takes place under atmospheric pressure. Foamless filling is preferably achieved by filling the container with counterpressure. Furthermore, in an alternative embodiment, filling is also possible via a shut-off pump or by means of a control valve and a calming section to prevent foaming. Preferably, a container with a spray ball is provided for cleaning. In a preferred embodiment, the degassing module comprises measuring devices for a specified filling of the container. Valves are used to release the excess pressure. In a preferred embodiment, a back pressure control with gradual release to atmospheric pressure can take place in order to further reduce foam formation. After degassing, the sample is transferred to a filtration device or a filter for removing suspended matter.

In an alternative embodiment, degassing is possible by ultrasonic treatment or directly by centrifugation. In case of centrifugation, the centrifuge is the degassing module, as degassing takes place during centrifugation. The centrifuge is preferably a vertical decanter centrifuge. Preferably, the extracted beer sample is led into a separation chamber of the centrifuge drum via an inlet pipe and accelerated therein. According to the invention, speeds between 600 and 4000, preferably between RCF 600 and 1200 RCF can be used. The non-liquid suspended matter or solids settle on the drum wall during centrifugation. Higher speeds can result in the pellets no longer detaching from the drum wall, which is not desired. It is therefore advantageous to select the speed in the above-mentioned range between 600 and 1200 RCF, whereby the duration of centrifugation must also be considered. The invention also includes intermediate values of the above-mentioned ranges. A centrifugation speed of about 800 RCF and a centrifugation time of at least 240 seconds have proven to be particularly advantageous.

According to the invention, centrifugal decantation and thus sedimentation takes place due to a difference in density of the individual phases of the beer suspension, which separate due to the centrifugal force. The density of solids is generally higher, so that they are flattened radially along the rotating drum shell. The lighter liquid phase, on the other hand, is concentrated in the center towards the drum's axis of rotation. A solids separator is preferably provided for an automated inline process. Preferably, a screw conveyor can also be used, which rotates at a slightly higher speed (differential speed) than the drum in order to convey the sedimented solids to the conical constriction of the drum. The solids are ejected from the drum through the top opening and are discharged downwards. By changing the differential speed, the residence time of the solids in the drum can be adjusted accordingly. The liquid part, however, flows along the screw flight to the cylindrical end of the bowl, where it is discharged via a transverse plate. It is then collected in an outer housing and discharged.

After the non-liquid suspended matter has been removed from the degassed and filtered beer sample either by filtration or centrifugation, the filtrate or centrifugate of the beer sample is fed into a sample conversion module. This consists of one or more sample containers, into each of which a defined sample volume of the filtered or centrifuged beer sample is fed. The basis for this is a so-called head-space analysis, i.e., extraction of the gas phase in the head area of the container for later measurement. Each sample container receives a defined volume of the beer sample. At the same time, heat is applied during incubation. By tempering the sample containers, a part of the liquid sample is transferred from the liquid phase to the gas phase. The thermal treatment also converts precursors of the VDK, e.g., 2-acetolactate to diacetyl (butane-2,3-dione) or 2-acetohydroxy-butyrate to pentane-2,3-dione. Here, an equilibrium is formed between the liquid phase and the gas phase. Heating in the sample container preferably takes place at a temperature of about 60° C., alternatively at a temperature between 60° C. and 90° C., preferably at about 80° C. The beer samples are heated in the sample container or containers until the precursors of the aroma components have been almost completely converted into the aroma components.

After the desired equilibration time, a representative quantity of gas is extracted from the sample container as a gas sample and is then analyzed. The volume of the beer sample fed into a sample container should preferably be precisely adjustable so that representative and reproducible measurement samples from the gas phase are available.

The sample containers are preferably made of stainless steel. In alternative embodiments, containers made of glass or plastic can also be used. Hydrophobic surfaces are preferred. Connections for fluid lines and lines for cleaning are provided on each container. There are also safety devices, for example to prevent liquid from entering the gas line or to prevent overpressure. CIP (“Cleaning In Place”)—capable containers and an automatic sample feed are also part of a preferred embodiment.

Each container comprises a heating device to achieve the desired conversion temperature of 60° C. or 80° C. for example. In order to accelerate the conversion of the precursors of the VDK to the VDK, it is additionally provided in a preferred embodiment that the beer sample is shaken or jarred in the sample containers. Shaking or jarring the sample is intended to support the conversion of the precursors and the achievement of the equilibrium state in the headspace of the container. Furthermore, the surface area at the phase boundary is increased, which supports diffusion across the phase boundary. Mixing also increases diffusion in the liquid. Preferably, the sample conversion module therefore also comprises an agitator that shakes the samples at frequencies preferably between >0 Hz and 20 Hz. Preferably, the agitator comprises an electric motor with reversal of the direction that drives a turntable bearing. In alternative embodiments, linear drives that are driven at high frequency are also possible. Circular motion with the aid of several linear drives and suitable controls is also possible. Alternatively, shaking baths, laboratory agitators or ultrasonic baths can also be used.

In the next process step, a sample gas is extracted from the head space of the sample container, which is then fed into a measuring device. Specifically, the sample gas is sent to a GC column via a sample loop. The sample should always have the same conditions, regardless of the pressure conditions in the sample container. A carrier gas, preferably nitrogen or synthetic air, is used to transport the sample gas. When the gas sample is transported, pressure initially builds up in the sample container due to conditioning. A valve can be used to release the pressure in the direction of the sample loop. By raising the liquid level in the sample container, the sample is conveyed in the direction of the sample loop, into which it flows under atmospheric pressure. Alternatively, pumps or auto-samplers can also be used to convey gas samples. In another alternative embodiment, the sample gas is extracted from the headspace, for example using a syringe or by applying a vacuum.

In a preferred embodiment, a fluid, i.e., a gas or liquid, is fed into the sample container to increase the pressure in the headspace of the sample container, so that an overpressure is created in the headspace. When feeding additional sample liquid into the sample container, the liquid level in the container rises, increasing the gas pressure in the headspace.

In the case of GC-IMS, the analysis usually takes 15 minutes, so that six samples can be measured in 90 minutes with one measuring system. With a large number of measuring devices, the turnover is of course accordingly increased by the multipliers.

Gas lines are provided for gas transport in order to conduct the sample gas from the headspace of the sample container to the measuring device, preferably GC-IMS. The gas lines are also suitable for blowing out or blowing dry empty sample containers and for overpressure protection.

The sample gas is separated by gas chromatography in a first process step and subjected to ion mobility spectroscopy (GC-IMS) in a second process step. This results in a two-dimensional separation, which also makes it possible to analyze a complex multi-component mixture such as beer. A particular advantage of this analysis method is that it can perform fully automated sample management, i.e., the analysis of the sample gases extracted from the sample containers for the qualitative and quantitative determination of vicinal diketones and other aroma components. Alternatively, other methods can also be used for the qualitative and quantitative determination of the aroma components, for example UV/VIS, GC-ECD (gas chromatography with electron capture detector) or GC-MS (gas chromatography with mass spectrometer). In GC-IMS, nitrogen or a nitrogen-containing gas mixture has proven to be advantageous as a carrier gas. Other carrier gases are also possible, such as helium.

Preferably, the amount of total diacetyl is determined, i.e., the sum of the free diacetyl and the α-acetolactate. The desired degree of maturation of the beer or the end point of fermentation is by all means achieved when the measured aroma components from the sample of the sample conversion module are below a desired threshold. The exact reference value depends on the respective beer type. Usually, the concentration of total diacetyl plus precursors (acetohydroxy acids) should not exceed 0.10 ppm. Butane-2,3-dione accounts for <0.05 ppm and pentane-2,3-dione for <0.02 ppm of this. Due to the processing of the samples according to the invention and the automated GC-IMS measurement, it is possible to achieve reference values of 1 ppb, i.e., aroma components, in particular vicinal diketones, with a content of 0.001 ppm. Until now, such specific monitoring of the beer fermentation and maturation processes was not possible in an in-line process.

Preferably, the interval and/or the number of measurements is/are performed depending on the presence and/or quantity of the examined components. When determining the end point, factors such as fermentation process, beer type and/or reaching a defined threshold are also included in the measurement intervals and number of measurements. In most cases, it is sufficient to limit the measurement to the vicinal diketones, i.e., total diacetyl and pentane-2,3-dione.

To determine the quality, a large number of components are analyzed, preferably using UV/VIS spectroscopy. The result is a spectroscopic signature that can be evaluated in a multivariate manner, i.e., in the form of a principal component analysis. Principal component analysis (PCA) is a mathematical method of multivariate statistics. PCA is used to structure, simplify and visualize extensive data sets by approximating a large number of statistical variables by a smaller number of linear combinations (the principal components) that are as convincing as possible. Such signatures are characteristic like a fingerprint for the respective beer and allow drawing conclusions about the quality of the beer.

With the method according to the invention, lead substances or marker compounds can be determined quickly, efficiently and fully automatically in real time, both qualitatively and quantitatively, in order to draw conclusions about the fermentation success and the degree of maturation of the respective batch. Faulty batches can thus be identified in real time without delays. The end points of fermentation or the degree of maturity can be determined very precisely for each individual batch in order to accurately record and ensure the quality of the beer.

The method according to the invention also makes it possible to replace the time-consuming laboratory analysis and shift it into the process. Alternatively, it is also possible to take and measure random samples for an existing laboratory analysis as a manual sample.

In addition to the method, the invention also comprises a plant for automated quality monitoring of the fermentation process and/or for determining the end point of fermentation during the beer brewing process in the production of beer by determining one or more aroma components or other ingredients of the beer, comprising:
a) a sampling device for the automated extraction of a beer sample from one or more fermentation vessels of a brewing plant,
b) a separation device, comprising a filter or a centrifuge, for degassing and/or removing non-liquid suspended matter in the beer sample,
c) a sample conversion module, consisting of one or more heatable sample containers, for converting precursors of the aroma components into the aroma components or for converting ingredients,
d) a measuring device for determining the aroma components or ingredients contained in the gas phase of the headspace of a sample container.

The separation device of the plant can perform two tasks. If the liquid is largely clear, there is no need to remove solids. In this case, for example, degassing would take place via centrifugation. However, if the beer sample is very cloudy and therefore has a high concentration of suspended matter, it is necessary to purify the beer sample by removing non-liquid suspended matter, i.e., solids, from it. Preferably, a degassing module for degassing the beer sample extracted from the fermentation vessel(s) is provided upstream of the separation device. Only then does the degassed beer sample enter the separation device, i.e., the filtration device or centrifuge.

In a preferred embodiment of the system, process media valves are provided for sampling, which are switched in particular to provide fresh water, hot water or steam for sterilization and for the supply of CIP media. Furthermore, in a preferred embodiment, there is also the option for a manual sample or for calibration.

The filter of the filtration module can preferably be backflushed after each measurement. In a preferred embodiment, a UV/VIS module is also provided for a spectroscopic multivariate evaluation of the beer's ingredients. This enables spectroscopic measurement of the filtered and degassed beer. Multivariate data analysis can also be performed to determine the quantity of higher alcohols, amino acids, sugars, esters or other compounds. Usually, however, the VDK load should be <500 ppb so that the UV/VIS produces suitable signatures. The process of the fermentation can be detected using multivariate statistics and a so-called “golden batch” can be determined for each batch. Furthermore, deviations from this average value can also be used to detect faulty batches. Ultimately, this reduces production times and increases quality.

In a preferred embodiment, the UV/VIS also enables the CIP processes to be monitored. particularly with regard to the composition of the cleaning media, but also with regard to the cleanness of water, alkalis and acids. In doing so, the CIP process management can be optimized reducing the use of water and chemicals and ultimately optimizing the cleaning. The UV/VIS module consists of a spectrometer with measuring cells plus the necessary equipment for data processing, software and electronics. It also includes the fluid technology for measuring and controlling the fluid flows. Instead of a UV/VIS, sensors could also be used to perform pH measurements, density measurements or electrical conductivity measurements, for example.

The sampling module is composed as described above and comprises one or more sample containers, which are preferably shaken by an agitator and heated by a heating device. The system according to the invention also includes the control technology and software for managing and controlling valves, pumps and sensors. The control technology monitors and starts flushing sequences when the starting conditions are met. Furthermore, problems in the components to be controlled can also be detected in order to trigger corresponding alarms. The entire fermentation process can be controlled and monitored, in particular the times of sampling, measurement and stopping of the fermentation process depending on the type of beer to be measured.

The supply of heat and the shaking of the sample containers of the sample conversion module ensure that a complete conversion of the precursors of the VDK to the VDK takes place. If the measured value of the sample extracted from the sample conversion module is below the desired reference value, it is ensured that the desired degree of maturation has been reached in the storage tanks.

The method according to the invention makes it possible to use the measurement results to specifically influence the brewing process, in particular if parameters fall below or exceed a defined threshold.

### BRIEF DESCRIPTION OF THE DRAWINGS

FIG. 1 shows a diagram of the method and system for automated monitoring of the fermentation success and the degree of maturation of beer during the brewing process.

FIG. 2 shows the development of total diacetyl during fermentation.

FIG. 3 shows a principal component analysis of three different fermentation samples.

FIG. 4 shows the spectra of a beer sample after vertical centrifugation.

### WAYS OF CARRYING OUT THE INVENTION

The invention is explained in more detail in the following example.

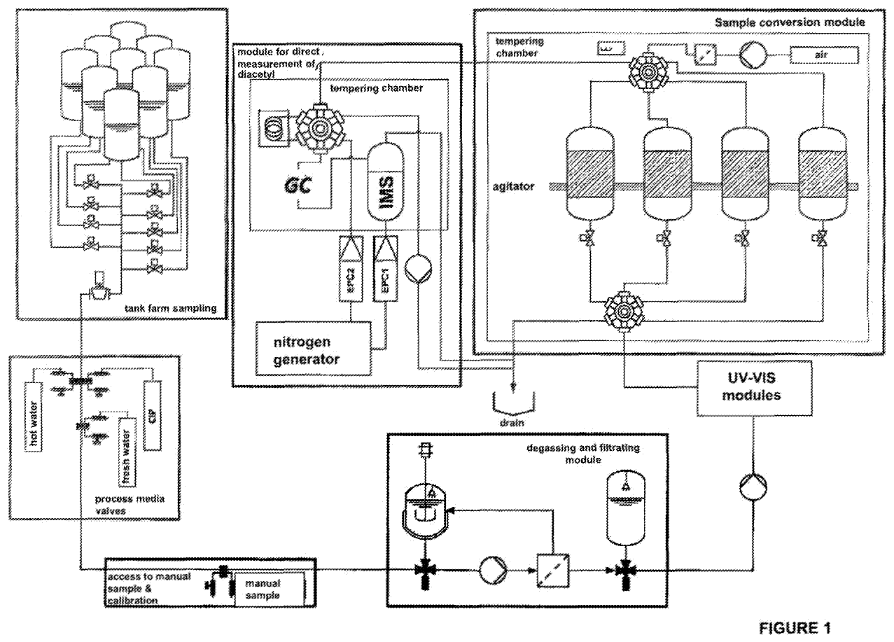

In FIG. 1, a tank farm consisting of several fermentation vessels can be seen at the top left. Beer samples can be extracted from individual or several vessels at specific, predetermined times. Alcoholic fermentation takes place in the fermentation vessels over several days. The breakdown of sugar by the yeasts produces alcohol and carbon dioxide. The precursors of the VDK are also produced during this process. Towards the end of the fermentation process, the VDK are converted into less flavor-intensive molecules. The object is for the beer to remain in the tanks for only as long as necessary in order to achieve a higher turnover in beer production.

By extracting samples from the tank, samples from the fermentation tank are automatically extracted in a set cycle by switching several valves. The automation eliminates the need for manual sampling. The sample is transferred from the tanks to the degassing and filtration module via process media valves. Sampling valves and sample lines are provided on each fermentation vessel. These ultimately lead to a leak-proof three-way/two-way valve through which the media can be fed into the system. Depending on the structure of the facility, the process media valves can supply the entire structure of the system or, alternatively, each module can have its own process media valves. The sample extracted from the tank farm is finally fed into the degassing and filtration module. First, degassing takes place, i.e., the beer sample is transported into the degassing module by pre-pressure with minimal foaming and the vessel is then depressurized,

Preferably, the degassing container contains a spray ball for cleaning and an agitator for degassing. Furthermore, measuring devices are also provided for specified filling of the container. A valve prevents overpressure. In a preferred embodiment, back pressure control with gradual release to atmospheric pressure is possible, with the aim of reducing foam formation. Ultimately, the degassing module enables low-foaming filling. A filtration module, i.e., a filter device with which it is possible to remove particles larger than 2 μm, is provided to further remove suspended matter. Filtration is necessary in order to obtain a clear and reproducible filtrate for subsequent measurement. It is also essential that there is no cross-contamination between the samples. The filter can be backflushed after each measurement. Optionally, the degassed and filtered sample can now be fed into a UV/VIS module for spectroscopic analysis.

Otherwise, the sample is directly fed into the sample conversion module. There, the sample is divided equally between the individual sample containers. The sample containers are heated to a specific temperature between preferably 50° C. and 90° C., preferably to a temperature of about 60° C. or about 80° C., for a specific period of time.

In a preferred embodiment, the samples are simultaneously shaken evenly by an agitator. Frequencies between >0 Hz-20 Hz are provided. The shaking of the sample supports the conversion of the precursors of the VDK and the achievement of the equilibrium state in the head space of the sample container. Some of the samples can also be led via a valve to a drain into which discarded media are also discharged, for example CIP or rinsing media. The sample conversion module also includes a temperature control chamber and devices for supplying air. Sample gas is taken from the head space of the sample container and fed into a measuring device. In the embodiment shown, this is a module for measuring diacetyl. The GC-IMS is used in the embodiment shown. The measuring module consists of electrical pressure controllers (EPC 1, EPC 2), a GC column for the first separation and an ion mobility spectrometer (IMS) for the second separation. A pump, sample loops and fluid lines as well as suitable valves and control components ensure automated measurement of the aroma components, in this case the determination of diacetyl.

In the embodiment shown, a module for direct diacetyl measurement is used as the measuring device. However, the invention is not limited to the determination of vicinal diketones, but can also be used for the qualitative and quantitative determination of other aroma components, i.e., young beer bouquet substances or bouquet substances.

The sample gas from the sample conversion module is driven through a carrier gas to pressurize the GC column. This is located in a temperature chamber. Depending on the mobility and the vapor pressure, the molecules in the column separate and emerge from the column as different batches. The separation efficiency and the time of exit can be controlled via the column type, the column length and the temperature. In the subsequent IMS, the incoming molecules are ionized and drawn through the carrier gas by an electric field. During preparation, a pump flushes the sample loop with air, as well as the EPCs, the GC column and the IMS with carrier gas. The pump is switched off during sample delivery. The EPC 1 pressure regulator allows the carrier gas (N₂) to flow through the IMS. The GC column is flushed via the EPC 2 pressure regulator. During sampling, the sample is fed into the sample loop via the fluid line. Once the sample is filled with a representative sample, the 6-port valve is switched and the sample is transferred to the GC column. Once sampling is complete, the valve switches back and the sample loop is flushed with air again using the pump.

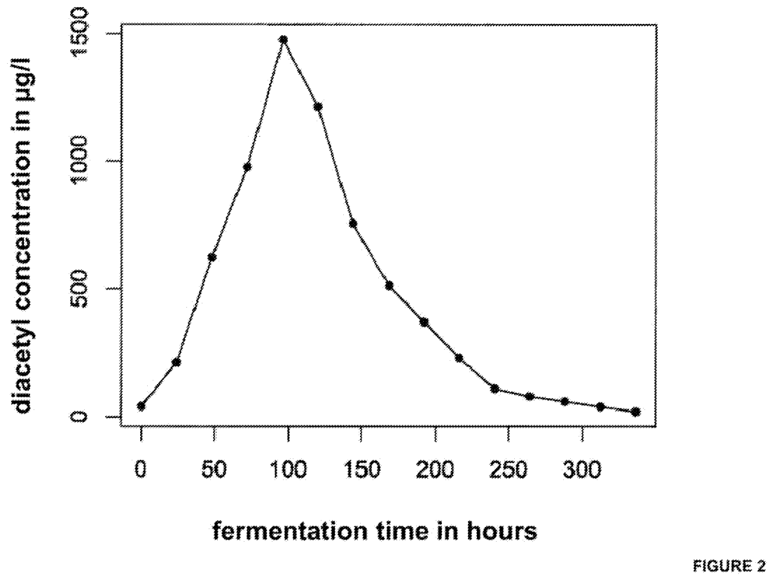

FIG. 2 shows an evaluation of a beer sample according to the invention based on a determination of total diacetyl in order to determine the end point of fermentation. It can be seen how the concentration of diacetyl decreases as the beer matures. The peak is at a fermentation time of about 90 hours. After that, the amount of total diacetyl decreases. After around 220 hours, the curve flattens out, i.e., the decrease is less rapid. When fermentation is stopped depends on the type of beer and the desired residual concentration of total diacetyl. For light lager beers, the threshold for diacetyl is usually around 5 ppb. For dark beers, a threshold between 40 and 60 ppb is sufficient, as the human sensory system would not be able to detect the flavor at even lower values.

Once the desired reference value has been reached, the maturation process is complete and the beer can be removed from the tank farm for bottling. The sample conversion in the sample conversion module, the associated increase in temperature and the optional shaking of the sample ensure that no further VDK can form in significant quantities. The beer retains its desired quality and is bottled neither too early nor too late to produce the finished beer.

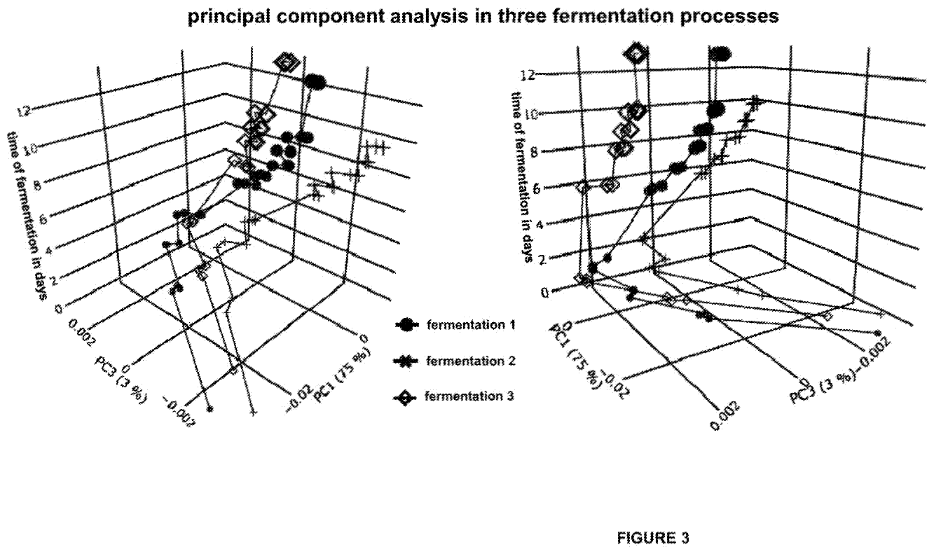

FIG. 3 shows a principal component analysis (“Principal Component Analysis”-PC) of three fermentation samples.

The principal component analysis is a multivariate statistical method. It is used to structure, simplify and visualize extensive data sets by approximating a large number of statistical variables by a smaller number of linear combinations that are as convincing as possible.

The three fermentation samples are analyzed in three-dimensional representation using a first principal component (PC1) and a second principal component (PC3) depending on the fermentation time (fermentation time in days). Fermentation sample 1 shows the development of diacetyl of an ideal sample. In comparison, fermentation samples 2 and 3 show a different curve, so that it can be concluded that the end point of fermentation has already been exceeded (fermentation 2) or has not yet been reached yet (fermentation 3). Ideally, the end point of fermentation is reached when the curve follows the course of the reference curve (fermentation 1).

According to the invention, the sample was analyzed by a sample preparation, i.e., the sample was extracted from a fermentation vessel and first degassed in a degassing module. Non-liquid suspended matter was then removed using a backflushable filter and the resulting filtrate was fed into a sample container. Here, the sample was incubated for about 90 minutes at a temperature of 60° C. The sample gas was then extracted from the headspace of the sample container and fed into a GC-IMS measuring device to determine the total acetyl concentration. This led to the result shown in FIG. 3.

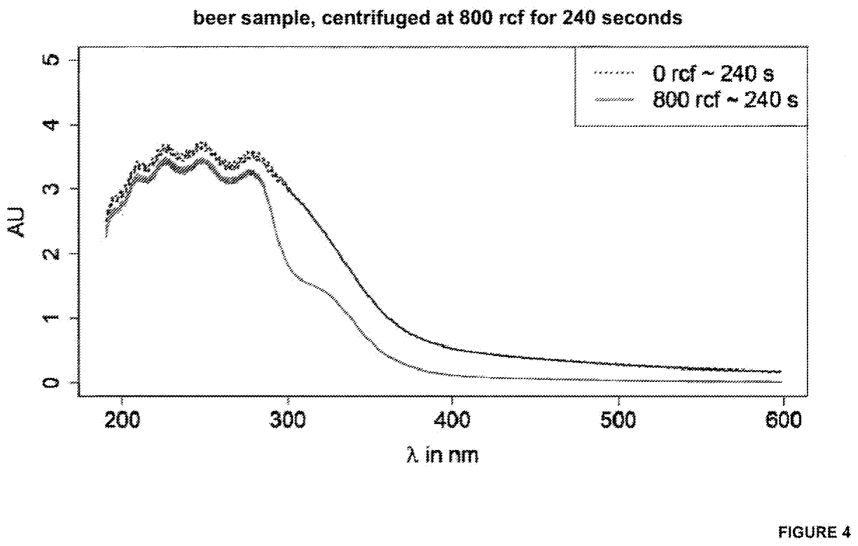

FIG. 4 shows the use of vertical decanter centrifuge for a beer sample. The centrifugation was performed at 800 RCF for a period of 240 seconds. A sample that was not centrifuged (0 RCF) served as a control. The adsorption units (AU=Absorbance Units) in the analyzed wavelength range are clearly below the control samples. The results were confirmed by a turbidity measurement.

1. A method for automated quality monitoring of the fermentation process and/or for determining the end point of fermentation during the beer brewing process in the production of beer by determining one or more aroma components or other ingredients of the beer, comprising the steps:
a. automated extracting of a beer sample from one or more fermentation vessels,
b. if required, degassing and filtering or centrifuging of the beer sample to remove non-liquid suspended matter,
c. feeding the filtrate or centrifugate of the beer sample into a sample conversion module consisting of one or more sample containers, into each of which a defined sample volume of the filtered or centrifuged beer sample is fed as required, which has sufficient headspace volume,
d. heating the samples in the sample container or containers to convert the precursors of the aroma components into aroma components or to convert ingredients, wherein the heating is performed until the precursors of the aroma components have been almost completely converted into the aroma components,
e. extracting sample gas from the headspace of the sample container or from one of the sample containers and transferring the sample gas to a measuring device for the qualitative and quantitative determination of one or more components of the beer.

2. The method for automated quality monitoring of the fermentation process and/or for determining the end point of fermentation during the beer brewing process in the production of beer according to claim 1, characterized in that the aroma components are vicinal diketones, preferably diacetyl (butane-2,3-dion) or pentane-2,3-dion.

3. The method for automated quality monitoring of the fermentation process and/or for determining the end point of fermentation during the beer brewing process in the production of beer according to claim 1, characterized in that the one or more sample containers of the sample conversion module with the samples divided therein are heated and simultaneously shaken.

4. The method for automated quality monitoring of the fermentation process and/or for determining the end point of fermentation during the beer brewing process in the production of beer according to claim 1, characterized in that before feeding the filtrate or centrifugate into the sample container(s), a spectroscopic measurement of the filtered and degassed beer sample, preferably via UV/VIS, is performed.

5. The method for automated quality monitoring of the fermentation process and/or for determining the end point of fermentation during the beer brewing process in the production of beer according to claim 1, characterized in that the measuring device is GC-IMS, GC-ECD, GC-MS, or an UV/VIS-spectrometer.

6. The method for automated quality monitoring of the fermentation process and/or for determining the end point of fermentation during the beer brewing process in the production of beer according to claim 1, characterized in that the fermentation process is evaluated by a principal component analysis of the spectroscopic signature of the selected components of the beer.

7. The method for automated quality monitoring of the fermentation process and/or for determining the end point of fermentation during the beer brewing process in the production of beer according to claim 1, characterized in that the exact end point of fermentation is reached when the measured components of the beer are below a desired threshold in the beer sample.

8. The method for automated quality monitoring of the fermentation process and/or for determining the end point of fermentation during the brewing process in the production of beer according to claim 1, characterized in that the sample gas is taken from the headspace of the sample container by pumping, introducing a fluid or suction.

9. The method for automated quality monitoring of the fermentation process and/or for determining the end point of fermentation during the beer brewing process in the production of beer according to claim 1, characterized in that, prior to filtration, the beer sample extracted from the fermentation vessel or vessels is transferred to a degassing module and is degassed.

10. The method for automated quality monitoring of the fermentation process and/or for determining the end point of fermentation during the beer brewing process in the production of beer according to claim 1, characterized in that the centrifugation is performed in a vertical decanter centrifuge.

11. The method for automated quality monitoring of the fermentation process and/or for determining the end point of fermentation during the beer brewing process in the production of beer according to claim 10, characterized in that the centrifugation takes place between 600 and 4000, preferably between 600 and 1200, preferred at approximately 800 RCF.

12. A plant for automated quality monitoring of the fermentation process and/or for determining the end point of fermentation during the beer brewing process in the production of beer by determining one or more aroma components or other ingredients of the beer, comprising:
a. a sampling device for the automated extraction of a beer sample from one or more fermentation vessels of a brewing plant,
b. a separation device, comprising a filter or a centrifuge, for degassing and/or removing non-liquid suspended matter in the beer sample as required,
c. a sample conversion module, consisting of one or more heatable sample containers, for converting precursors of aroma components into aroma components or for converting ingredients,
d. a measuring device for determining the aroma components or ingredients contained in the gas phase of the headspace of a sample container.

13. The plant for automated quality monitoring of the fermentation process and/or for determining the end point of fermentation during the beer brewing process in the production of beer according to claim 12, further comprising a degassing module for degassing the beer sample extracted from the fermentation vessel(s) before filtration in the separation device.

14. The plant for automated quality monitoring of the fermentation process and/or for determining the end point of fermentation during the beer brewing process in the production of beer according to claim 12, characterized in that the centrifugate of the separation device is a vertical decanter centrifuge.

15. The plant for automated quality monitoring of the fermentation process and/or for determining the end point of fermentation during the beer brewing process in the production of beer according to claim 12, characterized in that the process media valves are provided for providing fresh water, hot water or steam for sterilization as well as CIP media.

16. The plant for automated quality monitoring of the fermentation process and/or for determining the end point of fermentation during the beer brewing process in the production of beer according to claim 12, characterized in that the filter of the filtration module is backflushable after every measurement.
